SEQUENCE LISTING (16 sequences)

SEQ ID NO: 1 - protein, 25 aa, Artificial Sequence
  Synthesized
DIVLTQSPASLAVSLGQRATISCKA

SEQ ID NO: 2 - protein, 16 aa, Artificial Sequence
  Synthesized
NWFQQKPGQPPKLLIY

SEQ ID NO: 3 - protein, 32 aa, Artificial Sequence
  Synthesized
GIPARFSGSGSGTDFTLNIHPVEEEDAATYYC

SEQ ID NO: 4 - protein, 12 aa, Artificial Sequence
  Synthesized
TFGGGTKLEIKR

SEQ ID NO: 5 - protein, 25 aa, Artificial Sequence
  Synthesized
DVQLVESGGGLVQPGGSRKLSCAAS

SEQ ID NO: 6 - protein, 14 aa, Artificial Sequence
  Synthesized
WVRQAPEKGLEWVA

SEQ ID NO: 7 - protein, 31 aa, Artificial Sequence
  Synthesized
GRFTISRDNPKNTLFLQMTSLRSEDTAMYYC

SEQ ID NO: 8 - protein, 13 aa, Artificial Sequence
  Synthesized
DYWGQGTSVTVSS

SEQ ID NO: 9 - protein, 106 aa, Artificial Sequence
  Synthesized
ADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 324 aa, Artificial Sequence
  Synthesized
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 112 aa, Artificial Sequence
  Synthesized
DIVLTQSPASLAVSLGQRATISCKASQSIDYDGDSFMNWFQQKPGQPPKLLIYDASNVESGIPARFSGSGSGTDFTLNIHPVEEEDAATYYCQHSNEDPYTFGGGTKLEIKR

SEQ ID NO: 12 - protein, 114 aa, Artificial Sequence
  Synthesized
DVQLVESGGGLVQPGGSRKLSCAASGVTFSTFAMHWVRQAPEKGLEWVAYLNSASNLIYYADTLKGRFTISRDNPKNTLFLQMTSLRSEDTAMYYCTRHAMDYWGQGTSVTVSS

SEQ ID NO: 13 - protein, 218 aa, Artificial Sequence
  Synthesized
DIVLTQSPASLAVSLGQRATISCKASQSIDYDGDSFMNWFQQKPGQPPKLLIYDASNVESGIPARFSGSGSGTDFTLNIHPVEEEDAATYYCQHSNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 438 aa, Artificial Sequence
  Synthesized
DVQLVESGGGLVQPGGSRKLSCAASGVTFSTFAMHWVRQAPEKGLEWVAYLNSASNLIYYADTLKGRFTISRDNPKNTLFLQMTSLRSEDTAMYYCTRHAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 25 aa, Artificial Sequence
  Synthesized
EVQLVESGGDFVQPGGSRKLSCAAS

SEQ ID NO: 16 - protein, 324 aa, Artificial Sequence
  Synthesized
TKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2023/0391853 A1
(43) Pub. Date: Dec. 7, 2023

(54) Antibody against Novel Coronavirus, and Reagent and Kit for Detecting Novel Coronavirus

(71) Applicant: FAPON BIOTECH INC., Shenzhen, Guangdong (CN)

(72) Inventors: Peng CUI, Dongguan, Guangdong (CN); Zhiqiang HE, Dongguan, Guangdong (CN); Yuan MENG, Dongguan, Guangdong (CN); Dongmei ZHONG, Dongguan, Guangdong (CN); Wenjuan LOU, Dongguan, Guangdong (CN); Lingyun FAN, Dongguan, Guangdong (CN)

(21) Appl. No.: 18/034,394

(22) PCT Filed: Sep. 10, 2021
(86) PCT No.: PCT/CN2021/117802

§ 371 Date: Apr. 28, 2023

(30) Foreign Application Priority Data
Oct. 29, 2020 (CN) 202011182628.X

(51) Int. Cl. C07K 16/10; G01N 33/569; C12N 15/63

(52) U.S. Cl. CPC C07K 16/1003; G01N 33/56983; C12N 15/63; C07K 2317/565; C07K 2317/92; C07K 2317/567; G01N 2333/165; G01N 2469/10

(57) ABSTRACT

An antibody against a novel coronavirus, and a reagent and a kit for detecting the novel coronavirus, which relate to the technical field of antibodies. The antibody against the novel coronavirus includes a heavy chain complementarity determinant region and a light chain complementarity determinant region. The antibody has good affinity for N protein of the novel coronavirus, and has good sensitivity and specificity for detecting the novel coronavirus. Antibody options are provided for detection of the novel coronavirus.

### CROSS-REFERENCE TO RELATED APPLICATION

The present disclosure is a National Stage of International Patent Application No. PCT/CN2021/117802, filed on Sep. 10, 2021, and claims priority to Chinese Patent Application No. 202011182628.X, filed to the China National Intellectual Property Administration on Oct. 29, 2020 and entitled “Antibody against Novel Coronavirus, and Reagent and Kit for Detecting Novel Coronavirus”, the disclosure of which is hereby incorporated by reference in its entirety.

### SEQUENCE LISTING

The instant disclosure contains a Sequence Listing which has submitted electronically in ASCII format and is hereby incorporated by reference in its entirety. Said ASCII copy is named PN208322 SEQ LIST.txt and is 17,596 bytes in size. The sequence listing contains 16 sequences, which is identical in substance to the sequences disclosed in the PCT application, except the comments for the artificial sequences have been added, and includes no new matter.

### TECHNICAL FIELD

The present disclosure relates to the technical field of antibodies, and specifically, to an antibody against novel coronavirus, and a reagent and kit for detecting the novel coronavirus.

### BACKGROUND

A structural protein of the novel coronavirus 2019 novel coronavirus (2019-nCoV) is classified into spike glycoprotein (S protein), envelope glycoprotein (E protein), membrane glycoprotein (M protein) and nucleocapsid protein (N protein), and these proteins include a plurality of antigen epitopes. The N protein intertwines with viral genome RNAto form a viral nucleocapsid, which plays an important role in the synthesis of virus RNA. In addition, the N protein is relatively conserved and makes up the largest proportion of the structural proteins of the virus; and high levels of antibodies against the N protein can be produced in the body early in the infection. Finally, the N protein is an important marker protein of the novel coronavirus. Using the principle of specific binding of antigens and antibodies, the presence of the antigens may be detected by an N protein monoclonal antibody, so as to directly prove the presence of the novel coronavirus in a sample and achieving the detection of the novel coronavirus.

Antibodies to be detected are mainly classified into two categories, which are IgM and IgG. Currently, there is a lack of systematic studies on the production and duration of these two types of antibodies to the novel coronavirus. Generally, IgM antibodies are produced early, and once infected, the antibodies are produced quickly, maintained for a short period of time, and disappear quickly. A positive test in the blood may reflect that the body is in an acute state of infection, which may be used as an indicator of early infection. Compared with a nucleic acid testing method, samples of an antibody test are serum or plasma, and are less affected by sample sampling, thereby facilitating early diagnosis and exclusion of suspicious cases; and in addition, the test is rapid, convenient and suitable for large-scale screening.

Since the outbreak of the novel coronavirus 2019-nCoV pneumonia, it has spread globally, posing a serious threat to human life safety and health. Respiratory droplets and close contact transmission are the main transmission routes of the novel coronavirus pneumonia, and the potential for aerosol transmission exists in relatively closed environments with prolonged exposure to high concentration of aerosol. The 2019-nCoV is highly infectious, and most patients develop clinical symptoms after infection, but some patients are asymptomatic infections. Common signs in people infected with coronavirus include respiratory symptoms, fever, cough, shortness of breath, and difficult breathing. In more severe cases, the infection may lead to pneumonia, severe acute respiratory syndrome, renal failure, and even death. Although there is no specific treatment for the disease caused by the novel coronavirus, treatment of mild or asymptomatic patients may greatly improve the cure rate and shorten treatment time. Therefore, the detection or identification of the patients becomes particularly important.

At present, nucleic acid testing and viral gene sequencing are mainly used as confirmatory evidence of pathogenesis, and nucleic acid testing is subject to false negative results due to various factors such as sampling, operation and reagents. The detection rate of positive viral nucleic acid in patients with highly suspected 2019 novel coronavirus (2019-nCoV) infection is only 30%-50%. In addition, nucleic acid testing requires high requirements for instruments and equipment, testing sites and environmental conditions, and has disadvantages of long testing time and low throughput, not facilitating large-scale testing of the population under the current epidemic. Therefore, there is an urgent need to develop a rapid and convenient detection kit for clinical detection, to isolate the infected population as soon as possible, so as to stop the spread of the virus. Therefore, antibody detection kits have become more important.

There are few 2019-nCoV N protein monoclonal antibody products with varied performance.

### SUMMARY

The present disclosure provides an antibody or a functional fragment thereof against a novel coronavirus or an N protein thereof. The antibody or the functional fragment thereof includes the following complementarity determinant regions:
CDR-VH1: G-X1-T-F-S-X2-F-X3-M-H, wherein X1 is V or F, X2 is S or T, and X3 is G or A;
CDR-VH2: Y-X1-N-S-X2-S-N-X3-1-Y-Y-A-D-T-X4-K, wherein X1 is L or I, X2 is G or A, X3 is I, V or L, and X4 is 1, V or L;
CDR-VH3: X1-R-H-X2-M, wherein X1 is A or T, and X2 is A or V;
CDR-VL1: S-Q-S-X1-D-Y-X2-G-D-S-X3-M, wherein X1 is I, V or L, X2 is D or N, and X3 is F or Y;
CDR-VL2: X1-A-S-N-X2-E-S, wherein X1 is Aor D, and X2 is I, V or L; and
CDR-VL3: Q-X1-S-N-E-X2-P-Y, wherein X1 is N, H or Q, and X2 is D or E.

In some implementations,
in CDR-VH1, X1 is F;
in CDR-VH2, X1 is I;
in CDR-VH3, X1 is A; and
in CDR-VL1, X3 is Y.

In some implementations, in CDR-VH1, X2 is S.

In some implementations, in CDR-VH1, X2 is T.

In some implementations, in CDR-VH1, X3 is G.

In some implementations, in CDR-VH1, X3 is A.

In some implementations, in CDR-VH2, X2 is G.

In some implementations, in CDR-VH2, X2 is A.

In some implementations, in CDR-VH2, X3 is I.

In some implementations, in CDR-VH2, X3 is V.

In some implementations, in CDR-VH2, X3 is L.

In some implementations, in CDR-VH2, X4 is I.

In some implementations, in CDR-VH2, X4 is V.

In some implementations, in CDR-VH2, X4 is L.

In some implementations, in CDR-VH3, X2 is A.

In some implementations, in CDR-VH3, X2 is V.

In some implementations, in CDR-VL1, X1 is I.

In some implementations, in CDR-VL1, X1 is V.

In some implementations, in CDR-VL1, X1 is L.

In some implementations, in CDR-VL1, X2 is D.

In some implementations, in CDR-VL1, X2 is N.

In some implementations, in CDR-VL2, X1 is A.

In some implementations, in CDR-VL2, X1 is D.

In some implementations, in CDR-VL2, X2 is I.

In some implementations, in CDR-VL2, X2 is V.

In some implementations, in CDR-VL2, X2 is L.

In some implementations, in CDR-VL1, X1 is N.

In some implementations, in CDR-VL1, X1 is H.

In some implementations, in CDR-VL1, X1 is Q.

In some implementations, in CDR-VL2, X2 is D.

In some implementations, in CDR-VL2, X2 is E.

In some implementations, each complementarity determinant region of the antibody or the functional fragment thereof is selected from any one of the following mutation combinations 1-68:

| | CDR-VH1 | CDR-VH2 | CDR-VH3 | CDR-VL1 | CDR-VL2 | CDR-VL3 |
|---|---|---|---|---|---|---|
| | X2/X3 | X2/X3/X4 | X2 | X1/X2 | X1/X2 | X1/X2 |
| Mutation combination 1 | T/A | A/L/L | A | I/D | D/V | H/D |
| Mutation combination 2 | T/G | A/V/I | V | V/D | D/L | H/E |
| Mutation combination 3 | S/A | A/I/V | V | L/D | D/I | N/D |
| Mutation combination 4 | S/G | G/V/L | A | I/N | A/V | N/E |
| Mutation combination 5 | T/A | G/I/I | V | V/N | A/L | Q/D |
| Mutation combination 6 | S/G | A/I/V | A | L/N | A/I | Q/E |
| Mutation combination 7 | T/G | G/L/L | A | I/N | A/L | Q/D |
| Mutation combination 8 | T/A | G/V/L | V | V/D | D/V | N/D |
| Mutation combination 9 | S/A | A/L/V | V | L/N | A/I | H/D |
| Mutation combination 10 | T/A | G/I/L | A | I/D | D/L | Q/E |
| Mutation combination 11 | T/A | A/L/V | A | V/N | A/V | N/E |
| Mutation combination 12 | T/G | A/L/I | V | L/D | D/I | H/E |
| Mutation combination 13 | T/G | A/I/I | V | I/D | D/L | Q/E |
| Mutation combination 14 | T/G | A/L/V | A | L/N | A/V | H/D |
| Mutation combination 15 | T/G | G/L/L | A | V/D | D/V | N/E |
| Mutation combination 16 | S/G | G/L/L | V | V/N | A/L | Q/D |
| Mutation combination 17 | S/G | G/V/L | V | L/D | D/I | H/E |
| Mutation combination 18 | S/A | G/L/I | A | I/N | A/I | N/D |
| Mutation combination 19 | T/G | G/I/I | A | V/D | D/I | N/D |
| Mutation combination 20 | T/G | G/V/V | V | I/N | D/L | Q/E |
| Mutation combination 21 | S/A | A/L/V | V | L/D | D/V | H/D |
| Mutation combination 22 | T/G | G/V/L | A | V/N | A/I | N/E |
| Mutation combination 23 | S/A | A/V/L | A | I/D | A/L | Q/D |
| Mutation combination 24 | T/G | A/L/L | V | L/N | A/V | H/E |
| Mutation combination 25 | S/G | A/V/I | V | L/D | D/L | Q/E |
| Mutation combination 26 | T/G | G/V/V | A | I/N | A/L | N/D |
| Mutation combination 27 | T/G | A/I/L | A | V/D | D/V | H/E |
| Mutation combination 28 | S/A | G/L/I | V | L/N | A/V | Q/D |
| Mutation combination 29 | S/G | G/V/I | V | I/D | D/I | N/E |
| Mutation combination 30 | T/A | A/V/V | A | V/N | A/I | H/D |
| Mutation combination 31 | T/A | G/L/V | A | I/D | A/V | N/D |
| Mutation combination 32 | T/G | G/V/I | V | L/N | D/L | N/E |
| Mutation combination 33 | T/A | G/I/I | V | V/D | A/L | H/D |
| Mutation combination 34 | S/G | G/I/V | A | V/N | D/I | H/E |
| Mutation combination 35 | T/A | G/L/I | A | L/D | A/I | Q/D |
| Mutation combination 36 | S/G | G/L/V | V | I/N | D/V | Q/E |
| Mutation combination 37 | S/G | A/I/I | V | I/N | D/I | N/E |
| Mutation combination 38 | T/G | G/I/V | A | V/D | D/L | Q/D |
| Mutation combination 39 | T/G | G/V/L | A | L/N | D/V | H/E |
| Mutation combination 40 | T/G | G/L/L | V | I/D | A/I | N/D |
| Mutation combination 41 | T/A | G/L/L | V | V/N | A/L | Q/E |
| Mutation combination 42 | S/G | G/I/V | A | L/D | A/V | H/D |
| Mutation combination 43 | T/A | A/I/I | A | I/D | A/L | Q/E |
| Mutation combination 44 | S/G | G/V/L | V | I/N | D/V | H/D |
| Mutation combination 45 | T/A | A/V/V | V | L/D | A/I | N/E |
| Mutation combination 46 | S/A | A/I/L | A | L/N | D/L | Q/D |
| Mutation combination 47 | S/G | G/I/V | A | V/D | A/V | H/E |
| Mutation combination 48 | S/A | G/V/V | V | V/N | D/I | N/D |
| Mutation combination 49 | T/G | A/I/L | V | L/D | D/I | N/D |
| Mutation combination 50 | S/A | G/I/V | A | I/N | D/L | Q/E |
| Mutation combination 51 | S/A | G/V/V | A | V/D | D/V | H/D |
| Mutation combination 52 | S/G | A/I/I | V | L/N | A/I | N/E |
| Mutation combination 53 | T/A | A/L/V | V | I/D | A/L | Q/D |
| Mutation combination 54 | T/A | A/I/I | A | V/N | A/V | H/E |
| Mutation combination 55 | S/A | A/V/L | A | L/N | D/L | Q/E |
| Mutation combination 56 | T/G | G/V/V | V | L/D | A/V | N/D |
| Mutation combination 57 | S/A | A/V/I | V | V/N | D/V | H/E |
| Mutation combination 58 | T/G | A/I/V | A | V/D | A/L | Q/D |
| Mutation combination 59 | S/G | A/I/I | A | I/N | D/I | N/E |
| Mutation combination 60 | T/G | G/V/I | V | I/D | A/I | H/D |
| Mutation combination 61 | S/G | G/L/V | V | V/N | A/V | Q/E |
| Mutation combination 62 | T/A | A/L/V | A | L/D | D/L | Q/D |
| Mutation combination 63 | T/G | G/I/V | V | I/N | A/V | N/D |
| Mutation combination 64 | S/G | A/V/I | V | V/D | D/V | H/D |
| Mutation combination 65 | T/A | G/L/V | A | L/N | A/L | Q/E |
| Mutation combination 66 | S/G | A/I/I | A | I/D | D/I | N/E |
| Mutation combination 67 | T/G | G/V/I | V | V/N | A/I | H/E |
| Mutation combination 68 | T/G | A/I/V | A | L/D | D/L | Q/D |

In some implementations, the antibody or the functional fragment thereof binds to an N protein of novel coronavirus with an affinity of K_{D}≤8×10⁻⁹ mol/L. In some implementations, K_{D}≤7×10⁻¹⁰ mol/L.

In some implementations,
in CDR-VH1, X1 is V;
in CDR-VH2, XX is L;
in CDR-VH3, X1 is T; and
in CDR-VL1, X3 is F.

In some implementations, each complementarity determinant region of the antibody or the functional fragment thereof is selected from any one of the following mutation combinations 69-76:

| | CDR-VH1 | CDR-VH2 | CDR-VH3 | CDR-VL1 | CDR-VL2 | CDR-VL3 |
|---|---|---|---|---|---|---|
| | X2/X3 | X2/X3/X4 | X2 | X1/X2 | X1/X2 | X1/X2 |
| Mutation combination 69 | T/A | A/L/L | A | I/D | D/V | H/D |
| Mutation combination 70 | T/G | G/L/I | V | I/N | D/V | Q/D |
| Mutation combination 71 | S/A | G/V/L | A | L/N | D/V | N/D |
| Mutation combination 72 | S/G | G/L/L | A | V/N | A/V | Q/D |
| Mutation combination 73 | S/G | A/L/L | V | L/D | D/L | Q/E |
| Mutation combination 74 | S/G | G/I/L | V | L/D | D/L | H/E |
| Mutation combination 75 | T/A | A/L/I | A | L/D | A/L | H/D |
| Mutation combination 76 | T/G | G/I/L | V | V/D | A/I | N/E |

In some implementations, the antibody includes light chain framework regions FR1-L, FR2-L, FR3-L and FR4-L respectively successively having at least 80% identity to sequences SEQ ID NO:1, 2, 3 and 4, and/or heavy chain framework regions FR1-H, FR2-H, FR3-H and FR4-H respectively successively having at least 80% identity to sequences 5, 6, 7 and 8.

In some implementations, an amino acid sequence of FR1-H is shown as SEQ ID NO:15.

In some implementations, the antibody further includes a constant region.

In some implementations, the constant region is selected from any one of IgG1, IgG2, IgG3, IgG4, IgA, IgM, IgE and IgD.

In some implementations, the species of the constant region is derived from cattle, horses, dairy cattle, pigs, sheep, goats, rats, mice, dogs, cats, rabbits, camels, donkeys, deer, minks, chickens, ducks, geese, turkeys, game fowls or humans.

In some implementations, the constant region is derived from the mice.

In some implementations, the sequence of a light chain constant region of the constant region is shown as SEQ ID NO:9 or has at least 80% identity, and the sequence of a heavy chain constant region of the constant region is shown as SEQ ID NO:10 or has at least 80% identity.

In some implementations, the sequence of the heavy chain constant region is shown as SEQ ID NO:16.

In some implementations, the functional fragment is selected from any one of VHH, F(ab′)2, Fab′, Fab, Fv and scFv in the antibody.

The present disclosure provides a reagent or kit for detecting novel coronavirus or an N protein thereof. The reagent or kit includes any one of the above antibodies or the functional fragments thereof.

In some implementations, antibody or the functional fragment thereof is labeled with a detectable marker.

In some implementations, the detectable marker is selected from a fluorescent dye, an enzyme that catalyzes color development of substrates, radio isotope, a chemiluminescence reagent and a nanoparticle marker.

In some implementations, the fluorescent dye is selected from fluorescein dyes and derivatives thereof, rhodamine dyes and derivatives thereof, Cy series dyes and derivatives thereof, Alexa series dyes and derivatives thereof, and protein dyes and derivatives thereof.

In some implementations, the enzyme that catalyzes color development of substrates is selected from horseradish peroxidase, alkaline phosphatase, p-galactosidase, glucose oxidase, carbonic anhydrase, acetylcholinesterase and glucose 6-phosphate dehydrogenase.

In some implementations, the radio isotope is selected from ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ⁹⁴mTc, ⁹⁹mTc, ²⁰³Pb, ⁶⁷Ga, ⁶⁸Ga, ⁴³Sc, ⁴⁷Sc, ¹¹⁰mIn, ⁹⁷Ru, ⁶²Cu, ⁶⁴Cu, ⁶⁷Cu, ⁶⁸Cu, ⁸⁶Y, ⁸⁸Y, ¹²¹Sn, ¹⁶¹Tb, ¹⁶⁶Ho, ¹⁰⁵Rh, ¹⁷⁷Lu, ¹⁷²Lu and ¹⁸F.

In some implementations, the chemiluminescence reagent is selected from luminol and derivatives thereof, lucigenin, crustacean fluorescein and derivatives thereof, bipyridine ruthenium and derivatives thereof, acridinium ester and derivatives thereof, dioxycyclohexane and derivatives thereof, lophine and derivatives thereof, and peroxyoxalate and derivatives thereof.

In some implementations, the nanoparticle marker is selected from nanoparticles, colloids, organic nanoparticles, magnetic nanoparticles, quantum dot nanoparticles and rare earth complex nanoparticles.

In some implementations, the colloids are selected from colloidal metals, disperse dyes, dye labeled microspheres, and latexes.

In some implementations, the colloidal metals are selected from colloidal gold, colloidal silver and colloidal selenium.

The present disclosure provides a nucleic acid molecule. The nucleic acid molecule codes any one of the above antibodies or the functional fragments thereof.

The present disclosure provides a vector. The vector includes a nucleic acid fragment coding any one of the above antibodies or the functional fragments thereof.

The present disclosure provides a recombinant cell. The recombinant cell includes the vector.

The present disclosure provides an application of the antibody or the functional fragment thereof or the reagent or kit in detection of novel coronavirus.

The present disclosure provides an application of the antibody or the functional fragment thereof or the reagent or kit for detecting novel coronavirus.

The present disclosure provides a method for detecting novel coronavirus. The method includes the following operations.
A) Under conditions sufficient for a binding reaction to occur, any one of the above antibodies or the functional fragments thereof is in contact with a sample, so as to perform a binding reaction.
B) An immune complex produced by the binding reaction is detected.

The present disclosure provides a method for diagnosing a subject in a novel coronavirus infection or a disease associated with the novel coronavirus infection. The method includes the following operations.
A) Under conditions sufficient for a binding reaction to occur, any one of the above antibodies or the functional fragments thereof is in contact with a sample from the subject, so as to perform a binding reaction.
B) An immune complex produced by the binding reaction is detected.

In some implementations, the disease associated with the novel coronavirus infection includes respiratory symptoms, fever, cough, shortness of breath, difficult breathing, pneumonia, severe acute respiratory syndrome, and renal failure.

In some implementations, the subject in the novel coronavirus infection includes an asymptomatic patient, an infected patient without obvious symptoms, and a symptomatic infected patient.

The present disclosure provides a method for preparing any one of the above antibodies or the functional fragments thereof. The method includes: culturing the recombinant cell, and obtaining the antibody or the functional fragment from a culture product by means of separation and purification.

### BRIEF DESCRIPTION OF THE DRAWINGS

In order to illustrate the technical solutions in the embodiments of the present disclosure more clearly, the drawings used in the embodiments will be briefly introduced below. It is to be understood that the following drawings only illustrate some embodiments of the present disclosure, which therefore should not be regarded as limitations to the scope. For those of ordinary skill in the art, other related drawings may also be obtained in accordance with these drawings without creative efforts.

FIG. 1 shows reductive SDS-PAGE results of an antibody against novel coronavirus according to Embodiment 1.

### DETAILED DESCRIPTION OF THE EMBODIMENTS

In order to make the purposes, technical solutions and advantages of the embodiments of the present disclosure clearer, the technical solutions in the embodiments of the present disclosure will be clearly and completely described below. If specific conditions are not indicated in the implementations and embodiments, the implementations are carried out in accordance with the conventional conditions or the conditions recommended by manufacturers. Reagents or instruments used are conventional products that may be purchased commercially if the manufacturers are not specified.

Unless otherwise defined, all technical and scientific terms used herein have the same meaning as commonly understood by those of ordinary skill in the art to which the disclosure belongs. Although any methods and materials similar or equivalent to those described herein may be used in the practice or testing of preparations or unit doses herein, some methods and materials are now described. Unless otherwise stated, the technologies used or considered herein are standard methods. The materials, methods, and examples are illustrative only and not limiting.

Unless otherwise indicated, the present disclosure will be practiced by using conventional technologies of cell biology, molecular biology (including recombinant technologies), microbiology, biochemistry and immunology, and the conventional technologies are within the competence of those skilled in the art. The technologies are well explained in the literature, such as Molecular Cloning: A Laboratory Manual (2nd edition, Sambrook et. al., 1989); Oligonucleotide Synthesis (M. J. Gait, 1984); Animal Cell Culture (R. I. Freshney, 1987); Methods in Enzymology (Academic Press, Inc.); Handbook of Experimental Immunology (D. M. Weir and C. C. Blackwell); Gene Transfer Vectors for Mammalian Cells (J. M. Miller and M. P Calos, 1987); Current Protocols in Molecular Biology (F. M. Ausubel et. al., 1987); PCR: The Polymerase Chain Reaction (Mullis et. al., 1994); and Current Protocols in Immunology (J. E. Coligan et. al., 1991). Each of the literature is explicitly incorporated herein by reference.

### Term Definition

As used herein, the term “complementarity determinant region” means that an intact or complete antibody includes two heavy chains and two light chains. Each heavy chain includes a variable region (VH) and a constant region (CH); and each light chain includes a variable region (VL) and a constant region (CL). The antibody is in a “Y” shape, and the stem of Y consists of the second and third constant regions of two heavy chains bound together by means of disulfide bonds. Each arm of Y includes the variable region and the first constant region of the single heavy chain in combination with the variable region and the constant region of the single light chain. The variable region of the light chain and the variable region of the heavy chain are responsible for antigen binding. The variable regions in two chains generally include three highly variable regions, called complementarity determinant regions.

As used herein, when being used to represent an antibody, the term “functional fragment” refers to a portion of the antibody including a heavy or light chain polypeptide, and the polypeptide retains some or all of the binding activity of the antibody from which the fragment originated. These functional fragments may include (for example) Fd, Fv, Fab, F(ab′), F(ab)2, F(ab′)2, single-chain Fv (scFv), a double-chain antibody (diabody), a triple-chain antibody (triabody), a four-chain antibody (tetrabody), and a microbody (minibody). Other functional fragments may include (for example) heavy or light chain polypeptides, variable region polypeptides or CDR polypeptides or portions thereof, as long as these functional fragments retain the binding activity.

As used herein, the term “constant region” refers to the region of an antibody molecule near a C-terminal amino acid sequence that is relatively stable in both the light and heavy chains.

As used herein, the term “variable region” refers to the region of the antibody molecule near an N-terminal amino acid sequence that is highly variable in the light and heavy chains.

As used herein, the term “naked antibody stability” refers to an unlabeled antibody or a functional fragment thereof, such as an antibody or a functional fragment thereof that is not labeled with a detectable marker.

Some implementations of the present disclosure provide an antibody against novel coronavirus, and a reagent and kit for detecting novel coronavirus. The antibody may specifically bind to an N protein of the novel coronavirus, has high affinity for the N protein, and has good sensitivity and specificity for detecting the novel coronavirus. According to the present disclosure, a wider range of antibody options are provided for detection of the novel coronavirus.

Some implementations of the present disclosure provide an antibody or a functional fragment thereof against a novel coronavirus or an N protein thereof. The antibody or the functional fragment thereof includes the following complementarity determinant regions:
CDR-VH1: G-X1-T-F-S-X2-F-X3-M-H, wherein X1 is V or F, X2 is S or T, and X3 is G or A;
CDR-VH2: Y-X1-N-S-X2-S-N-X3-1-Y-Y-A-D-T-X4-K, wherein X1 is L or I, X2 is G or A, X3 is I, V or L, and X4 is 1, V or L;
CDR-VH3: X1-R-H-X2-M, wherein X1 is A or T, and X2 is A or V;
CDR-VL1: S-Q-S-X1-D-Y-X2-G-D-S-X3-M, wherein X1 is I, V or L, X2 is D or N, and X3 is F or Y;
CDR-VL2: X1-A-S-N-X2-E-S, wherein X1 is A or D, and X2 is I, V or L; and
CDR-VL3: Q-X1-S-N-E-X2-P-Y, wherein X1 is N, H or Q, and X2 is D or E.

The antibody or the functional fragment thereof against the novel coronavirus provided in the present disclosure includes the above complementarity determinant region structures. The above complementarity determinant region structures may make the antibody or the functional fragment thereof specifically bind to the N protein of the novel coronavirus, such that the antibody or the functional fragment thereof has high affinity for the N protein, and has good sensitivity and specificity for detecting the novel coronavirus. According to the present disclosure, a wider range of antibody options are provided for detection of the novel coronavirus.

In an optional implementation, in CDR-VH1, X1 is F; in CDR-VH2, X1 is I; in CDR-VH3, X1 is A; and in CDR-VL1, X3 is Y

Experimental results in this embodiment show that the antibody shows higher affinity for the N protein of the novel coronavirus when the above mutation sites in each complementarity determinant region are the above amino acid residues.

In an optional implementation, in CDR-VH1, X2 is S.

In an optional implementation, in CDR-VH1, X2 is T.

In an optional implementation, in CDR-VH1, X3 is G.

In an optional implementation, in CDR-VH1, X3 is A.

In an optional implementation, in CDR-VH2, X2 is G.

In an optional implementation, in CDR-VH2, X2 is A.

In an optional implementation, in CDR-VH2, X3 is I.

In an optional implementation, in CDR-VH2, X3 is V.

In an optional implementation, in CDR-VH2, X3 is L.

In an optional implementation, in CDR-VH2, X4 is I.

In an optional implementation, in CDR-VH2, X4 is V.

In an optional implementation, in CDR-VH2, X4 is L.

In an optional implementation, in CDR-VH3, X2 is A.

In an optional implementation, in CDR-VH3, X2 is V.

In an optional implementation, in CDR-VL1, X1 is I.

In an optional implementation, in CDR-VL1, X1 is V.

In an optional implementation, in CDR-VL1, X1 is L.

In an optional implementation, in CDR-VL1, X2 is D.

In an optional implementation, in CDR-VL1, X2 is N.

In an optional implementation, in CDR-VL2, X1 is A.

In an optional implementation, in CDR-VL2, X1 is D.

In an optional implementation, in CDR-VL2, X2 is I.

In an optional implementation, in CDR-VL2, X2 is V.

In an optional implementation, in CDR-VL2, X2 is L.

In an optional implementation, in CDR-VL1, X1 is N.

In an optional implementation, in CDR-VL1, X1 is H.

In an optional implementation, in CDR-VL1, X1 is Q.

In an optional implementation, in CDR-VL2, X2 is D.

In an optional implementation, in CDR-VL2, X2 is E.

In an optional implementation, each complementarity determinant region of the antibody or the functional fragment thereof is selected from any one of the following mutation combinations 1-68:

| | CDR-VH1 | CDR-VH2 | CDR-VH3 | CDR-VL1 | CDR-VL2 | CDR-VL3 |
|---|---|---|---|---|---|---|
| | X2/X3 | X2/X3/X4 | X2 | X1/X2 | X1/X2 | X1/X2 |
| Mutation combination 1 | T/A | A/L/L | A | I/D | D/V | H/D |
| Mutation combination 2 | T/G | A/V/I | V | V/D | D/L | H/E |
| Mutation combination 3 | S/A | A/I/V | V | L/D | D/I | N/D |
| Mutation combination 4 | S/G | G/V/L | A | I/N | A/V | N/E |
| Mutation combination 5 | T/A | G/I/I | V | V/N | A/L | Q/D |
| Mutation combination 6 | S/G | A/I/V | A | L/N | A/I | Q/E |
| Mutation combination 7 | T/G | G/L/L | A | I/N | A/L | Q/D |
| Mutation combination 8 | T/A | G/V/L | V | V/D | D/V | N/D |
| Mutation combination 9 | S/A | A/L/V | V | L/N | A/I | H/D |
| Mutation combination 10 | T/A | G/I/L | A | I/D | D/L | Q/E |
| Mutation combination 11 | T/A | A/L/V | A | V/N | A/V | N/E |
| Mutation combination 12 | T/G | A/L/I | V | L/D | D/I | H/E |
| Mutation combination 13 | T/G | A/I/I | V | I/D | D/L | Q/E |
| Mutation combination 14 | T/G | A/L/V | A | L/N | A/V | H/D |
| Mutation combination 15 | T/G | G/L/L | A | V/D | D/V | N/E |
| Mutation combination 16 | S/G | G/L/L | V | V/N | A/L | Q/D |
| Mutation combination 17 | S/G | G/V/L | V | L/D | D/I | H/E |
| Mutation combination 18 | S/A | G/L/I | A | I/N | A/I | N/D |
| Mutation combination 19 | T/G | G/I/I | A | V/D | D/I | N/D |
| Mutation combination 20 | T/G | G/V/V | V | I/N | D/L | Q/E |
| Mutation combination 21 | S/A | A/L/V | V | L/D | D/V | H/D |
| Mutation combination 22 | T/G | G/V/L | A | V/N | A/I | N/E |
| Mutation combination 23 | S/A | A/V/L | A | I/D | A/L | Q/D |
| Mutation combination 24 | T/G | A/L/L | V | L/N | A/V | H/E |
| Mutation combination 25 | S/G | A/V/I | V | L/D | D/L | Q/E |
| Mutation combination 26 | T/G | G/V/V | A | I/N | A/L | N/D |
| Mutation combination 27 | T/G | A/I/L | A | V/C | D/V | H/E |
| Mutation combination 28 | S/A | G/L/I | V | L/N | A/V | Q/D |
| Mutation combination 29 | S/G | G/V/I | V | I/D | D/I | N/E |
| Mutation combination 30 | T/A | A/V/V | A | V/N | A/I | H/D |
| Mutation combination 31 | T/A | G/L/V | A | I/D | A/V | N/D |
| Mutation combination 32 | T/G | G/V/I | V | L/N | D/L | N/E |
| Mutation combination 33 | T/A | G/I/I | V | V/D | A/L | H/D |
| Mutation combination 34 | S/G | G/I/V | A | V/N | D/I | H/E |
| Mutation combination 35 | T/A | G/L/I | A | L/D | A/I | Q/D |
| Mutation combination 36 | S/G | G/L/V | V | I/N | D/V | Q/E |
| Mutation combination 37 | S/G | A/I/I | V | I/N | D/I | N/E |
| Mutation combination 38 | T/G | G/I/V | A | V/D | D/L | Q/D |
| Mutation combination 39 | T/G | G/V/L | A | L/N | D/V | H/E |
| Mutation combination 40 | T/G | G/L/L | V | I/D | A/I | N/D |
| Mutation combination 41 | T/A | G/L/L | V | V/N | A/L | Q/E |
| Mutation combination 42 | S/G | G/I/V | A | L/D | A/V | H/D |
| Mutation combination 43 | T/A | A/I/I | A | I/D | A/L | Q/E |
| Mutation combination 44 | S/G | G/V/L | V | I/N | D/V | H/D |
| Mutation combination 45 | T/A | A/V/V | V | L/D | A/I | N/E |
| Mutation combination 46 | S/A | A/I/L | A | L/N | D/L | Q/D |
| Mutation combination 47 | S/G | G/I/V | A | V/D | A/V | H/E |
| Mutation combination 48 | S/A | G/V/V | V | V/N | D/I | N/D |
| Mutation combination 49 | T/G | A/I/L | V | L/D | D/I | N/D |
| Mutation combination 50 | S/A | G/I/V | A | I/N | D/L | Q/E |
| Mutation combination 51 | S/A | G/V/V | A | V/D | D/V | H/D |
| Mutation combination 52 | S/G | A/I/I | V | L/N | A/I | N/E |
| Mutation combination 53 | T/A | A/L/V | V | I/D | A/L | Q/D |
| Mutation combination 54 | T/A | A/I/I | A | V/N | A/V | H/E |
| Mutation combination 55 | S/A | A/V/L | A | L/N | D/L | Q/E |
| Mutation combination 56 | T/G | G/V/V | V | L/D | A/V | N/D |
| Mutation combination 57 | S/A | A/V/I | V | V/N | D/V | H/E |
| Mutation combination 58 | T/G | A/I/V | A | V/D | A/L | Q/D |
| Mutation combination 59 | S/G | A/I/I | A | I/N | D/I | N/E |
| Mutation combination 60 | T/G | G/V/I | V | I/D | A/I | H/D |
| Mutation combination 61 | S/G | G/L/V | V | V/N | A/V | Q/E |
| Mutation combination 62 | T/A | A/L/V | A | L/D | D/L | Q/D |
| Mutation combination 63 | T/G | G/I/V | V | I/N | A/V | N/D |
| Mutation combination 64 | S/G | A/V/I | V | V/D | D/V | H/D |
| Mutation combination 65 | T/A | G/L/V | A | L/N | A/L | Q/E |
| Mutation combination 66 | S/G | A/I/I | A | I/D | D/I | N/E |
| Mutation combination 67 | T/G | G/V/I | V | V/N | A/I | H/E |
| Mutation combination 68 | T/G | A/I/V | A | L/D | D/L | Q/D |

In an optional implementation, the antibody or the functional fragment thereof binds to an N protein of the novel coronavirus with an affinity of K_{D}≤8×10⁻⁹ mol/L.

In an optional implementation, K_{D}≤7×10⁻¹⁰ mol/L.

In an optional implementation, K_{D}≤8×10⁻⁹ mol/L, K_{D}≤7×10⁻⁹ mol/L, K_{D}≤6×10⁻⁹ mol/L, K_{D}≤5×10⁻⁹ mol/L, K_{D}≤4×10⁻⁹ mol/L, K_{D}≤3×10⁻⁹ mol/L, K_{D}≤2×10⁻⁹ mol/L, K_{D}≤1×10⁻⁹ mol/L, K_{D}≤9×10⁻¹⁰ mol/L, K_{D}≤8×10⁻¹⁰ mol/L, K_{D}≤7×10⁻¹⁰ mol/L, K_{D}≤6×10⁻¹⁰ mol/L, K_{D}≤5×10⁻¹⁰ mol/L, K_{D}≤4×10⁻¹⁰ mol/L, K_{D}≤3×10⁻¹⁰ mol/L, K_{D}≤2×10⁻¹⁰ mol/L, K_{D}≤1×10⁻¹⁰ mol/L, K_{D}≤9×10⁻¹¹ mol/L or K_{D}8×10⁻¹¹ mol/L.

In an optional implementation, 8.75×10⁻¹¹ mol/L≤K_{D}≤7.08×10⁻¹⁰ mol/L.

K_{D} is detected by referring to the method in the embodiments of the present disclosure.

In an optional implementation, in CDR-VH1, X1 is V; in CDR-VH2, X1 is L; in CDR-VH3, X1 is T; and in CDR-VL1, X3 is F.

In an optional implementation, each complementarity determinant region of the antibody or the functional fragment thereof is selected from any one of the following mutation combinations 69-76:

| | CDR-VH1 | CDR-VH2 | CDR-VH3 | CDR-VL1 | CDR-VL2 | CDR-VL3 |
|---|---|---|---|---|---|---|
| | X2/X3 | X2/X3/X4 | X2 | X1/X2 | X1/X2 | X1/X2 |
| Mutation combination 69 | T/A | A/L/L | A | I/D | D/V | H/D |
| Mutation combination 70 | T/G | G/L/I | V | I/N | D/V | Q/D |
| Mutation combination 71 | S/A | G/V/L | A | L/N | D/V | N/D |
| Mutation combination 72 | S/G | G/L/L | A | V/N | A/V | Q/D |
| Mutation combination 73 | S/G | A/L/L | V | L/D | D/L | Q/E |
| Mutation combination 74 | S/G | G/I/L | V | L/D | D/L | H/E |
| Mutation combination 75 | T/A | A/L/I | A | L/D | A/L | H/D |
| Mutation combination 76 | T/G | G/I/L | V | V/D | A/I | N/E |

In an optional implementation, the antibody includes light chain framework regions FR1-L, FR2-L, FR3-L and FR4-L having sequences successively shown as SEQ ID NOs:1-4, and/or heavy chain framework regions FR1-H, FR2-H, FR3-H and FR4-H having sequences successively shown as SEQ ID NOs:5-8.

Generally, a variable region (VH) of the heavy chain and a variable region (VL) of the light chain may be obtained by connecting the following numbered CDRs and FRs in the following combination arrangement: FR1-CDR1-FR2-CDR2-FR3-CDR3-FR4.

In some implementations, the antibody includes light chain framework regions FR1-L, FR2-L, FR3-L and FR4-L respectively successively having at least 80% identity to sequences SEQ ID NO:1, 2, 3 and 4, and/or heavy chain framework regions FR1-H, FR2-H, FR3-H and FR4-H respectively successively having at least 80% identity to sequences SEQ ID NO: 5, 6, 7 and 8.

It is to be noted that, in other implementations and embodiments, the amino acid sequence of each framework region of the antibody or the functional fragment thereof provided in the present disclosure may have at least 80%, 81%, 82%, 83%, 84%, 85%, 86%, 87%, 88%, 89%, 90%, 91%, 92%, 93%, 94%, 95%, 96%, 97%, 98%, or 99% identity to that of the above corresponding framework region (SEQ ID NO:1, 2, 3, 4, 5, 6, 7 or 8). For example, an amino acid sequence of FR1-H may also be shown as SEQ ID NO:15.

In an optional implementation, the antibody further includes a constant region.

In an optional implementation, the constant region is selected from a constant region of any one of IgG1, IgG2, IgG3, IgG4, IgA, IgM, IgE and IgD.

In an optional implementation, the species source of the constant region is mammals or poultry animals. In an optional implementation, the mammals include cattle, horse, dairy cow, pig, sheep, goat, rat, mouse, dog, cat, rabbit, camel, donkey, deer, mink or humans. In an optional implementation, the poultry animals include chickens, ducks, geese, turkeys, or gamecocks.

In an optional implementation, the species source of the constant region is cattle, horse, dairy cow, pig, sheep, goat, rat, mouse, dog, cat, rabbit, camel, donkey, deer, mink, chicken, duck, goose, turkey, gamecock or human.

In an optional implementation, the constant region is derived from mouse.

In an optional implementation, the sequence of a light chain constant region (CL) of the constant region is shown as SEQ ID NO:9, and the sequence of a heavy chain constant region (CH) of the constant region is shown as SEQ ID NO:10.

It is to be noted that, in other embodiments, the sequence of the constant region of the antibody provided in the present disclosure may have at least 80%, 81%, 82%, 83%, 84%, 85%, 86%, 87%, 88%, 89%, 90%, 91%, 92%, 93%, 94%, 95%, 96%, 97%, 98%, or 99% identity to that of the above constant region (SEQ ID NO: 9 or 10). For example, the heavy chain constant region may also be shown as SEQ ID NO:16.

In an optional implementation, the functional fragment is selected from any one of VHH, F(ab′)2, Fab′, Fab, Fv and scFv of the antibody.

The functional fragment of the above antibody usually has the same binding specificity as its source antibody. It would have been readily understood by those skilled in the art according to content recorded in the present disclosure, the functional fragment of the above antibody may be obtained by means of methods including, for example, but not limited to, enzymatic digestion (including but not limited to pepsin or papain) and/or by means of a method of splitting disulfide bonds through chemical reduction. On the basis of an intact antibody structure provided in the present disclosure, the functional fragment would have been readily obtained by those skilled in the art.

The functional fragment of the antibody may also be obtained by means of recombinant genetic technologies also known to those skilled in the art or by means of synthesis, for example, by automated peptide synthesizers, such as those sold by, but not limited to, Applied BioSystems, etc. An implementation of the present disclosure provides a reagent or a kit for detecting a novel coronavirus or an N protein thereof. The reagent or the kit includes any one of the above antibodies or the functional fragments thereof.

In an optional implementation, the antibody or the functional fragment thereof in the reagent or kit is labeled with a detectable marker.

As used herein, the “detectable marker” refers to a class of substances having properties such as luminescence, color development, radioactivity, etc., that can be directly observed by naked eyes or detected or detected by an instrument, and by means of the properties, qualitative or quantitative detection of a corresponding target may be achieved.

In an optional implementation, the detectable marker includes, but is not limited to, a fluorescent dye, an enzyme that catalyzes color development of substrates, a radio isotope, a chemiluminescence reagent and a nanoparticle marker.

In an actual use process, those skilled in the art may choose the appropriate marker according to detection conditions or practical needs, regardless of the use of any marker, it is within the scope of protection of the present disclosure.

In an optional implementation, the fluorescent dye includes, but is not limited to, fluorescein dyes and derivatives thereof (for example, including, but not limited to, Fluorescein Isothiocyanate (FITC), hydroxyfluorescein (FAM) and Tetrachloro Fluorescein (TET) or analogues thereof), rhodamine dyes and derivatives thereof (for example, including, but not limited to, red rhodamine (RBITC), tetramethyl rhodamine (TAMRA) and rhodamine B (TRITC) or analogues thereof), Cy series dyes and derivatives thereof (for example, including, but not limited to, Cy2, Cy3, Cy3B, Cy3.5, Cy5 and Cy5.5 or analogues thereof), Alexa series dyes and derivatives thereof (for example, including, but not limited to, AlexaFluor350, 405, 430, 488, 532, 546, 555, 568, 594, 610, 633, 647, 680, 700 and 750 or analogues thereof), and protein dyes and derivatives thereof (for example, including, but not limited to, Phycoerythrin (PE), Phycocyanin (PC), Allophycocyanin (APC), polymethionin-Chlorophyll Protein (preCP)).

In an optional implementation, the enzyme that catalyzes color development of substrates includes, but is not limited to, horseradish peroxidase, alkaline phosphatase, p-galactosidase, glucose oxidase, carbonic anhydrase, acetylcholinesterase and glucose 6-phosphate dehydrogenase.

In an optional implementation, the radio isotope includes, but is not limited to, ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ⁹⁴mTc, ⁹⁹mTc, ²⁰³Pb, ⁶⁷Ga, ⁶⁸Ga, ⁴³Sc, ⁴⁷Sc, ¹¹⁰mIn, ⁹⁷Ru, ⁶²Cu, ⁶⁴Cu, ⁶⁷Cu, ⁶⁸Cu, ⁸⁶Y, ⁸⁸Y, ¹²¹Sn, ¹⁶¹Tb, ¹⁶⁶Ho, ¹⁰⁵Rh, ¹⁷⁷Lu, ¹⁷²Lu and ¹⁸F.

In an optional implementation, the chemiluminescence reagent includes, but is not limited to, luminol and derivatives thereof, lucigenin, a crustacean fluorescein and derivatives thereof, a bipyridine ruthenium and derivatives thereof, an acridinium ester and derivatives thereof, a dioxycyclohexane and derivatives thereof, lophine and derivatives thereof, and a peroxyoxalate and derivatives thereof.

In an optional implementation, the nanoparticle marker includes, but is not limited to, nanoparticles, colloids, organic nanoparticles, magnetic nanoparticles, quantum dot nanoparticles and rare earth complex nanoparticles.

In an optional implementation, the colloids include, but are not limited to, colloidal metals, disperse dyes, dye labeled microspheres, and latexes.

In an optional implementation, the colloidal metals include, but are not limited to, colloidal gold, colloidal silver and colloidal selenium. An implementation of the present disclosure provides a nucleic acid molecule encoding the above antibody or the functional fragment thereof. An implementation of the present disclosure provides a vector including a nucleic acid molecule. An implementation of the present disclosure provides a recombinant cell including the vector. An implementation of the present disclosure provides a method for preparing the antibody or the functional fragment thereof. The method includes: culturing the recombinant cell, and obtaining the antibody or the functional fragment from a culture product by means of separation and purification.

On the basis of the amino acid sequence of the antibody or the functional fragment thereof provided in the present disclosure, it would have been readily conceivable to those skilled in the art to prepare the antibody or the functional fragment thereof with a genetic engineering technology or other technologies (chemical synthesis and hybridoma cells). For example, the antibody or the functional fragment thereof is isolated and purified from a culture product of a recombinant cell capable of recombining and expressing any one of the above antibodies or the functional fragments thereof. It would have been readily achievable for those skilled in the art. Based on this, the antibody or the functional fragment thereof of the present disclosure is within the scope of protection of the present disclosure regardless of used preparation technologies.

### EMBODIMENTS

The characteristics and performance of the present disclosure are further described below in detail with reference to embodiments.

### Embodiment 1

In this embodiment, restriction endonuclease and Prime Star DNA polymerase were purchased from Takara. A MagExtractor-RNA extraction kit was purchased from TOYOBO. A BD SMART™ RACE cDNA Amplification Kit was purchased from Takara. A pMD-18T vector was purchased from Takara. A plasmid extraction kit was purchased from Tengen Corporation. Primer synthesis and gene sequencing were performed by Invitrogen.

1 Construction of Recombinant Plasmid

(1) Antibody Gene Preparation

mRNA was extracted from a hybridoma cell line secreting an antibody against an N protein of the novel coronavirus; a DNA product was obtained by means of RT-PCR; the product was inserted into a pMD-18T vector after rTaq DNA polymerase was used to perform an A tailing reaction, and was transformed into DH5α competent cells; and after colonies were grown, heavy chain and light chain gene clones were taken, and four clones each were sent to a gene sequencing company for sequencing.

(2) Sequence Analysis of Antibody Variable Region Genes

The gene sequences obtained from the above sequencing were placed in the IMGT antibody database (which was derived from http://www.imgt.org) for analysis, and VNTI 11.5 software was used for analysis, so as to determine that both heavy and light chain primers were correct for amplified genes. In the gene fragment amplified by the light chain primer pair, a VL gene sequence was 342 bp and belonged to a VkII gene family, there is a 57 bp leader peptide sequence in front of it; and in the gene fragment amplified by the heavy chain primer pair, a VH gene sequence was 336 bp and belonged to a VH1 gene family, there is a 57 bp leader peptide sequence in front of it.

(3) Construction of Recombinant Antibody Expression Plasmid

pcDNA™ 3.4 TOPO® vector was the constructed recombinant antibody eukaryotic expression vector. The polyclonal restriction sites such as HindIII, BamHI, and EcoRI have already been introduced into the expression vector, and is named as pcDNA3.4A expression vector, and then referred to as 3.4A expression vector; Based on the above sequencing results of antibody variable region genes in pMD-18T, specific primers for VL and VH genes for the antibody were designed with HindIII and EcoRI restriction sites and protection base at both ends, respectively, and a 0.72 kb light chain gene fragment and a 1.41 kb heavy chain gene fragment were amplified by means of PCR amplification.

The heavy chain and light chain gene fragments were double digested with HindIII/EcoRI; the 3.4A vector was double digested with the HindIII/EcoRI; the heavy chain gene and light chain gene were ligated into the 3.4A expression vector after the fragments and the vectors were purified and recycled, so as to obtain the recombinant expression plasmids for the heavy chain and the light chain.

2 Stable Cell Line Screening

(1) Transient Transfection of CHO Cells with Recombinant Antibody Expression Plasmids to Determine the Activity of Expression Plasmid

The plasmid obtained from steps 1-(3) was diluted with ultrapure water to 400 ng/ml, CHO cells were adjusted to 1.43×10⁷ cells/ml in a centrifuge tube, 100 μL of the plasmid and 700 μL of cells were mixed and transferred into an electroporation cup, electroporating, sampling and counting were performed on days 3, 5 and 7, and samples were collected for detection on day 7.

A 2019-nCoV N protein antigen was diluted with the coating liquid (with a main component NaHCO₃) to 1 μg/ml, with 100 μL per well, and stayed overnight at 4° C.; on the next day, it was washed twice with a washing solution, and patted dry; blocking solution (20% BSA+80% PBS) was added, 120 μL per well, and incubated at 37° C. for 1 h and patted dry; the diluted cell supernatant was added, 100 μL/well, and incubated at 37° C. for 30 min (partial supernatant 1 h); it was washed with the washing solution for 5 times and patted dry; goat anti-mouse IgG-HRP was added, 100 uL/well, 37° C., and 30 min; it was washed with the washing solution for 5 times, and patted dry; a color developing solution A (50 uL/well, containing citric acid+sodium acetate+acetanilide+urea peroxide) was added, and a color developing solution B (50 uL/well, containing EDTA-2Na+concentrated H₂SO₄) was added, 10 min; stop solution was added, 50 uL/well; and an OD value was read at 450 nm (refer to 630 nm) on a microplate reader. Results showed that the reaction OD was still greater than 1.0 after 1000-fold dilution of cell supernatant, and the reaction OD of the wells without cell supernatant was less than 0.1, indicating that the antibody produced after plasmid transient transfer was active against the 2019-nCoV N protein antigen.

(2) Linearization of Recombinant Antibody Expression Plasmid

Preparation of the following reagents: 50 μL of buffer, 100 μg/tube of DNA, 10 μL of Puv I enzyme, sterile water added up to 500 μL, enzyme digestion was performed overnight in a 37° C. water bath; extraction with equal volumes of phenol/chloroform/isoamyl alcohol (lower layer) 25:24:1 was performed firstly, followed with chloroform (aqueous phase) in sequence; and precipitation was performed with 0.1 times the volume (aqueous phase) of 3M sodium acetate and 2 times the volume of ethanol on ice, rinsing and precipitation was performed with 70% ethanol, organic solvents were removed, after the ethanol was completely evaporated, re-melting was performed with proper amount of sterilized water, and finally the concentration was determined.

(3) Stable Transfection of Recombinant Antibody Expression Plasmid, and Pressurized Screening of Stable Cell Lines

The plasmid obtained from steps 2-(2) was diluted to 400 ng/ml with ultrapure water, CHO cells were adjusted to 1.43×10⁷ cells/ml in a centrifuge tube, 100 μL plasmid and 700 μL cells were mixed and transferred into an electroporation cup, electroporating, counting was performed on the next day; and pressurized culture was performed for about 25 days in 25 umol/L MSX 96 well.

Cloned wells with cells were observed and labeled under a microscope, and confluence was recorded; culture supernatant was taken and samples were sent for detection; cell lines with antibody concentration and high relative concentration were selected to be transferred to 24 wells, and transferred to 6 wells in about 3 days; after 3 days of seeding and batch culture, cell density was adjusted to 0.5×10⁶ cells/ml, 2.2 ml was taken for batch culture, and cell density was adjusted to 0.3×10⁶ cells/ml, 2 ml was taken for seeding; 7-day 6-well batch culture supernatant was send for detection, and the cell lines with small antibody concentration and cell diameter were selected to be transferred in TPP for seed preservation and passage.

3 Recombinant Antibody Production

(1) Cell Expansion Culture

The cells obtained by means of passaging in steps 2-(3) were resuscitated and then cultured in a 125 ml shake flask, with an inoculum volume of 30 ml, and a culture medium was a 100% Dynamis culture medium, and was placed in a shaker at 120 r/min, 37° C., and 8% CO₂. Culture was performed for 72 h, inoculation expansion culture was performed with the inoculation density of 500,000 cells/ml, the volume of expansion culture was calculated according to production requirements, and the culture medium was the 100% Dynamis culture medium. Then, expansion culture was performed every 72 h. When a cell volume met the production requirements, the inoculation density was strictly controlled to about 500,000 cells/ml for production.

(2) Shake Flask Production and Purification

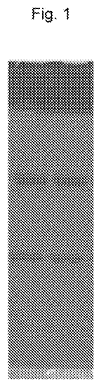

Shake flask parameters: 120 r/min, 37° C. and 8% CO₂. Fed-batch cultivation: material replenishment was started when cultured in the shake flask up to 72 h, HyClone Cell Boost Feed 7a was fed daily with 3% of an initial culture volume, and Feed 7b was fed daily with one thousandth of the initial culture volume until day 12 (material replenishment at day 12). Glucose was replenished with 3 g/L at day 6. Samples were collected at day 13. Affinity purification was performed with protein A affinity chromatography columns. 4 μg of the purified antibody was taken for reductive SDS-PAGE, and 4 μg of a foreign control antibody was used as a control, electrophoretogram was shown in FIG. 1 below and showed two bands after reductive SDS-PAGE, and one Mr was 50 KD (heavy chain, SEQ ID NO:14) and the other Mr was 28 KD (light chain, SEQ ID NO:13).

### Embodiment 2

Performance Detection of Antibody

(1) Activity Detection of the Antibody and Mutants Thereof in Embodiment 1

The sequence of the antibody (VT) in Embodiment 1 was analyzed, and the heavy chain variable region was shown as SEQ ID NO:12. Herein, the amino acid sequence of each complementarity determinant region in the heavy chain variable region was as follows.
CDR-VH1: G-V(X1)-T-F-S-T(X2)-F-A(X3)-M-H;
CDR-VH2: Y-L(X1)-N-S-A(X2)-S-N-L(X3)-1-Y-Y-A-D-T-L(X4)-K; and
CDR-VH3: T(X1)-R-H-A(X2)-M.

The light chain variable region was shown as SEQ ID NO:11. The amino acid sequence of each complementarity determinant region in the light chain variable region was as follows.
CDR1-VL: S-Q-S-I(X1)-D-Y-D(X2)-G-D-S-F(X3)-M;
CDR-VL2: D(X1)-A-S-N-V(X2)-E-S; and
CDR-VL3: Q-H(X1)-S-N-E-D(X2)-P-Y.

On the basis of the antibody (WT) against the novel coronavirus in Embodiment 1, mutations were made in the complementarity determinant regions for sites related to the activity of the antibody, herein X1, X2, X3, and X4 were mutation sites, as shown in Table 1 below.

TABLE 1
Mutation sites related to the activity of the antibodies.

| | CDR-VH1 | CDR-VH2 | CDR-VH3 | CDR-VL1 |
|---|---|---|---|---|
| | X1 | X1 | X1 | X3 |
| WT | V | L | T | F |
| Mutation 1 | F | I | A | Y |
| Mutation 2 | F | I | A | F |
| Mutation 3 | V | I | T | Y |
| Mutation 4 | F | L | A | F |
| Mutation 5 | V | I | A | F |

Detection on the binding activity of the antibody in Table 1:

A 2019-nCoV N protein antigen was diluted with the coating liquid (with a main component NaHCO₃) for microporous plate coating, with 100 μl per well, overnight at 4° C.; on the next day, it was washed twice with a washing solution, and patted dry; blocking solution (20% BSA+80% PBS) was added, 120 μL per well, and incubated at 37° C. for 1 h and patted dry; diluted monoclonal antibody in Table 1 was added, 100 μL/well, and incubated at 37° C. for 30 min-60 min; it was washed with the washing solution for 5 times and patted dry; goat anti-mouse IgG-HRP was added, 100 uL/well, 37° C., and 30 min; it was washed with the washing solution (PBS) for 5 times, and patted dry; a color development solution A (50 μl/well, containing 2.1 g/L of citric acid, 12.25 g/L of sodium acetate, 0.07 g/L of acetanilide, and 0.5 g/L of urea peroxide) was added, and then a color development solution B (50 μl/well, containing 1.05 g/L of citric acid, 0.186 g/L of EDTA·2Na, 0.45 g/L of TMB, and 0.2 ml/L of concentrated HCl) was added for 10 min; a stop solution (50 μl/well, containing 0.75 g of EDTA·2Na and 10.2 ml/L of concentrated H₂SO₄) was added; and an OD value was read at 450 nm (referring to 630 nm) on a microplate reader. Results were shown in Table 2 below.

TABLE 2
Activity data of WT antibody and mutants thereof

| Antibody concentration (ng/ml) | 5000 | 1000 | 500 | 250 | 125 | 0.00 |
|---|---|---|---|---|---|---|
| WT | 1.165 | 1.061 | 0.985 | 0.650 | 0.284 | 0.029 |
| Mutation 1 | 1.382 | 1.251 | 1.148 | 0.858 | 0.493 | 0.028 |
| Mutation 2 | 1.3014 | 1.2518 | 1.145 | 0.854 | 0.507 | 0.023 |
| Mutation 3 | 1.2201 | 1.296 | 1.195 | 0.802 | 0.501 | 0.025 |
| Mutation 4 | 1.284 | 1.291 | 1.156 | 0.864 | 0.484 | 0.022 |
| Mutation 5 | 1.204 | 1.130 | 1.052 | 0.743 | 0.358 | 0.090 |

The results in Table 2 showed that both the WT and the mutants had good binding activity, and mutation 1 had the optimal activity.

(2) Affinity Detection of the Antibody and Mutants Thereof

(a) On the basis of mutation 1, other sites in each CDR region were mutated, and the sequence of each mutation was shown in Table 3.

TABLE 3
Mutation site related to the affinity of an antibody

| | CDR-VH1 | CDR-VH2 | CDR-VH3 | CDR-VL1 | CDR-VL2 | CDR-VL3 |
|---|---|---|---|---|---|---|
| | X2/X3 | X2/X3/X4 | X2 | X1/X2 | X1/X2 | X1/X2 |
| Mutation 1 | T/A | A/L/L | A | I/D | D/V | H/D |
| Mutation 1-1 | T/G | A/V/I | V | V/D | D/L | H/E |
| Mutation 1-2 | S/A | A/I/V | V | L/D | D/I | N/D |
| Mutation 1-3 | S/G | G/V/L | A | I/N | A/V | N/E |
| Mutation 1-4 | T/A | G/I/I | V | V/N | A/L | Q/D |
| Mutation 1-5 | S/G | A/I/V | A | L/N | A/I | Q/E |
| Mutation 1-6 | T/G | G/L/L | A | I/N | A/L | Q/D |
| Mutation 1-7 | T/A | G/V/L | V | V/D | D/V | N/D |
| Mutation 1-8 | S/A | A/L/V | V | L/N | A/I | H/D |
| Mutation 1-9 | T/A | G/I/L | A | I/D | D/L | Q/E |
| Mutation 1-10 | T/A | A/L/V | A | V/N | A/V | N/E |
| Mutation 1-11 | T/G | A/L/I | V | L/D | D/I | H/E |
| Mutation 1-12 | T/G | A/I/I | V | I/D | D/L | Q/E |
| Mutation 1-13 | T/G | A/L/V | A | L/N | A/V | H/D |
| Mutation 1-14 | T/G | G/L/L | A | V/D | D/V | N/E |
| Mutation 1-15 | S/G | G/L/L | V | V/N | A/L | Q/D |
| Mutation 1-16 | S/G | G/V/L | V | L/D | D/I | H/E |
| Mutation 1-17 | S/A | G/L/I | A | I/N | A/I | N/D |
| Mutation 1-18 | T/G | G/I/I | A | V/D | D/I | N/D |
| Mutation 1-19 | T/G | G/V/V | V | I/N | D/L | Q/E |
| Mutation 1-20 | S/A | A/L/V | V | L/D | D/V | H/D |
| Mutation 1-21 | T/G | G/V/L | A | V/N | A/I | N/E |
| Mutation 1-22 | S/A | A/V/L | A | I/D | A/L | Q/D |
| Mutation 1-23 | T/G | A/L/L | V | L/N | A/V | H/E |
| Mutation 1-24 | S/G | A/V/I | V | L/D | D/L | Q/E |
| Mutation 1-25 | T/G | G/V/V | A | I/N | A/L | N/D |
| Mutation 1-26 | T/G | A/I/L | A | V/D | D/V | H/E |
| Mutation 1-27 | S/A | G/L/I | V | L/N | A/V | Q/D |
| Mutation 1-28 | S/G | G/V/I | V | I/D | D/I | N/E |
| Mutation 1-29 | T/A | A/V/V | A | V/N | A/I | H/D |
| Mutation 1-30 | T/A | G/L/V | A | I/D | A/V | N/D |
| Mutation 1-31 | T/G | G/V/I | V | L/N | D/L | N/E |
| Mutation 1-32 | T/A | G/I/I | V | V/D | A/L | H/D |
| Mutation 1-33 | S/G | G/I/V | A | V/N | D/I | H/E |
| Mutation 1-34 | T/A | G/L/I | A | L/D | A/I | Q/D |
| Mutation 1-35 | S/G | G/L/V | V | I/N | D/V | Q/E |
| Mutation 1-36 | S/G | A/I/I | V | I/N | D/I | N/E |
| Mutation 1-37 | T/G | G/I/V | A | V/D | D/L | Q/D |
| Mutation 1-38 | T/G | G/V/L | A | L/N | D/V | H/E |
| Mutation 1-39 | T/G | G/L/L | V | I/D | A/I | N/D |
| Mutation 1-40 | T/A | G/L/L | V | V/N | A/L | Q/E |
| Mutation 1-41 | S/G | G/I/V | A | L/D | A/V | H/D |
| Mutation 1-42 | T/A | A/I/I | A | I/D | A/L | Q/E |
| Mutation 1-43 | S/G | G/V/L | V | I/N | D/V | H/D |
| Mutation 1-44 | T/A | A/V/V | V | L/D | A/I | N/E |
| Mutation 1-45 | S/A | A/I/L | A | L/N | D/L | Q/D |
| Mutation 1-46 | S/G | G/I/V | A | V/D | A/V | H/E |
| Mutation 1-47 | S/A | G/V/V | V | V/N | D/I | N/D |
| Mutation 1-48 | T/G | A/I/L | V | L/D | D/I | N/D |
| Mutation 1-49 | S/A | G/I/V | A | I/N | D/L | Q/E |
| Mutation 1-50 | S/A | G/V/V | A | V/D | D/V | H/D |
| Mutation 1-51 | S/G | A/I/I | V | L/N | A/I | N/E |
| Mutation 1-52 | T/A | A/L/V | V | I/D | A/L | Q/D |
| Mutation 1-53 | T/A | A/I/I | A | V/N | A/V | H/E |
| Mutation 1-54 | S/A | A/V/L | A | L/N | D/L | Q/E |
| Mutation 1-55 | T/G | G/V/V | V | L/D | A/V | N/D |
| Mutation 1-56 | S/A | A/V/I | V | V/N | D/V | H/E |
| Mutation 1-57 | T/G | A/I/V | A | V/D | A/L | Q/D |
| Mutation 1-58 | S/G | A/I/I | A | I/N | D/I | N/E |
| Mutation 1-59 | T/G | G/V/I | V | I/D | A/I | H/D |
| Mutation 1-60 | S/G | G/L/V | V | V/N | A/V | Q/E |
| Mutation 1-61 | T/A | A/L/V | A | L/D | D/L | Q/D |
| Mutation 1-62 | T/G | G/I/V | V | I/N | A/V | N/D |
| Mutation 1-63 | S/G | A/V/I | V | V/D | D/V | H/D |
| Mutation 1-64 | T/A | G/L/V | A | L/N | A/L | Q/E |
| Mutation 1-65 | S/G | A/I/I | A | I/D | D/I | N/E |
| Mutation 1-66 | T/G | G/V/I | V | V/N | A/I | H/E |
| Mutation 1-67 | T/G | A/I/V | A | L/D | D/L | Q/D |

Affinity Analysis

An AMC sensor was used, the purified antibody was diluted to 10 μg/mL with phosphate tween buffer (PBST, with main components being Na₂HPO₄+NaCl+TW-20), and the 2019-nCoV N protein antigen was diluted with the PBST in gradients: 1.41 μg/mL, 0.70 μg/mL, 0.35 μg/mL, 0.18 μg/mL, 0.09 μg/mL, and 0.04 μg/mL.

Operational process: equilibrium was performed in buffer 1 (PBST) for 60 s, the antibody was cured in an antibody solution for 300 s, incubation was performed in buffer 2 (PBST) for 180 s, binding was performed in an antigen solution for 420 s, dissociation was performed in buffer 2 for 1200 s, sensor regeneration was performed with a 10 mM pH 1.69 GLY solution and buffer 3, and data were outputted. K_{D} represents an equilibrium dissociation constant, that was, the affinity; kon represents the binding rate; and kdis represents the dissociation rate. Results were shown in Table 4 below.

TABLE 4
Affinity detection data

| | K_{D}(M) | kon(1/Ms) | kdis(1/s) |
|---|---|---|---|
| Mutation 1 | 1.20E−10 | 1.82E+06 | 2.18E−04 |
| Mutation 1-1 | 1.28E−10 | 1.63E+06 | 2.08E−04 |
| Mutation 1-2 | 1.44E−10 | 2.15E+06 | 3.09E−04 |
| Mutation 1-3 | 1.09E−10 | 2.55E+06 | 2.77E−04 |
| Mutation 1-4 | 1.35E−10 | 1.90E+06 | 2.57E−04 |
| Mutation 1-5 | 1.38E−10 | 2.97E+06 | 4.09E−04 |
| Mutation 1-6 | 3.27E−10 | 1.97E+06 | 6.44E−04 |
| Mutation 1-7 | 2.65E−10 | 2.85E+06 | 7.55E−04 |
| Mutation 1-8 | 3.57E−10 | 2.05E+06 | 7.31E−04 |
| Mutation 1-9 | 1.84E−10 | 2.66E+06 | 4.90E−04 |
| Mutation 1-10 | 5.72E−10 | 1.16E+06 | 6.63E−04 |
| Mutation 1-11 | 8.75E−11 | 2.97E+06 | 2.60E−04 |
| Mutation 1-12 | 2.20E−10 | 2.92E+06 | 6.41E−04 |
| Mutation 1-13 | 1.51E−10 | 2.73E+06 | 4.13E−04 |
| Mutation 1-14 | 3.83E−10 | 1.25E+06 | 4.79E−04 |
| Mutation 1-15 | 4.24E−10 | 1.73E+06 | 7.33E−04 |
| Mutation 1-16 | 1.18E−10 | 2.48E+06 | 2.93E−04 |
| Mutation 1-17 | 1.78E−10 | 2.95E+06 | 5.26E−04 |
| Mutation 1-18 | 4.74E−10 | 1.16E+06 | 5.50E−04 |
| Mutation 1-19 | 1.61E−10 | 2.43E+06 | 3.92E−04 |
| Mutation 1-20 | 1.70E−10 | 1.56E+06 | 2.65E−04 |
| Mutation 1-21 | 7.08E−10 | 1.11E+06 | 7.86E−04 |
| Mutation 1-22 | 9.77E−11 | 2.22E+06 | 2.17E−04 |
| Mutation 1-23 | 1.29E−10 | 1.85E+06 | 2.38E−04 |
| Mutation 1-24 | 1.45E−10 | 2.98E+06 | 4.33E−04 |
| Mutation 1-25 | 9.19E−11 | 2.71E+06 | 2.49E−04 |
| Mutation 1-26 | 2.36E−10 | 2.41E+06 | 5.68E−04 |
| Mutation 1-27 | 3.25E−10 | 1.73E+06 | 5.63E−04 |
| Mutation 1-28 | 5.11E−10 | 1.47E+06 | 7.51E−04 |
| Mutation 1-29 | 4.37E−10 | 1.22E+06 | 5.33E−04 |
| Mutation 1-30 | 1.91E−10 | 2.97E+06 | 5.66E−04 |
| Mutation 1-31 | 1.96E−10 | 2.53E+06 | 4.97E−04 |
| Mutation 1-32 | 4.48E−10 | 1.66E+06 | 7.43E−04 |
| Mutation 1-33 | 2.17E−10 | 1.15E+06 | 2.49E−04 |
| Mutation 1-34 | 1.32E−10 | 2.03E+06 | 2.67E−04 |
| Mutation 1-35 | 1.55E−10 | 2.79E+06 | 4.33E−04 |
| Mutation 1-36 | 3.23E−10 | 1.49E+06 | 4.82E−04 |
| Mutation 1-37 | 2.49E−10 | 2.17E+06 | 5.40E−04 |
| Mutation 1-38 | 3.37E−10 | 2.17E+06 | 7.32E−04 |
| Mutation 1-39 | 4.74E−10 | 1.21E+06 | 5.74E−04 |
| Mutation 1-40 | 5.56E−10 | 1.28E+06 | 7.12E−04 |
| Mutation 1-41 | 2.63E−10 | 2.92E+06 | 7.67E−04 |
| Mutation 1-42 | 1.98E−10 | 2.46E+06 | 4.87E−04 |
| Mutation 1-43 | 2.05E−10 | 2.12E+06 | 4.34E−04 |
| Mutation 1-44 | 1.53E−10 | 2.36E+06 | 3.60E−04 |
| Mutation 1-45 | 1.74E−10 | 2.56E+06 | 4.45E−04 |
| Mutation 1-46 | 2.56E−10 | 2.26E+06 | 5.79E−04 |
| Mutation 1-47 | 2.19E−10 | 1.13E+06 | 2.47E−04 |
| Mutation 1-48 | 1.34E−10 | 2.88E+06 | 3.86E−04 |
| Mutation 1-49 | 5.12E−10 | 1.41E+06 | 7.22E−04 |
| Mutation 1-50 | 5.12E−10 | 1.44E+06 | 7.37E−04 |
| Mutation 1-51 | 2.08E−10 | 1.93E+06 | 4.01E−04 |
| Mutation 1-52 | 1.45E−10 | 2.10E+06 | 3.04E−04 |
| Mutation 1-53 | 1.99E−10 | 2.20E+06 | 4.37E−04 |
| Mutation 1-54 | 2.26E−10 | 2.99E+06 | 6.76E−04 |
| Mutation 1-55 | 1.83E−10 | 1.16E+06 | 2.12E−04 |
| Mutation 1-56 | 2.13E−10 | 1.33E+06 | 2.83E−04 |
| Mutation 1-57 | 3.09E−10 | 2.22E+06 | 6.85E−04 |
| Mutation 1-58 | 4.63E−10 | 1.72E+06 | 7.97E−04 |
| Mutation 1-59 | 2.90E−10 | 2.48E+06 | 7.18E−04 |
| Mutation 1-60 | 5.62E−10 | 1.25E+06 | 7.02E−04 |
| Mutation 1-61 | 1.66E−10 | 2.80E+06 | 4.64E−04 |
| Mutation 1-62 | 4.47E−10 | 1.60E+06 | 7.15E−04 |
| Mutation 1-63 | 2.86E−10 | 1.91E+06 | 5.46E−04 |
| Mutation 1-64 | 1.74E−10 | 2.86E+06 | 4.99E−04 |
| Mutation 1-65 | 1.25E−10 | 1.80E+06 | 2.25E−04 |
| Mutation 1-66 | 1.55E−10 | 2.53E+06 | 3.92E−04 |
| Mutation 1-67 | 6.65E−10 | 1.04E+06 | 6.92E−04 |

The data in Table 4 showed that both mutation 1 and series mutants thereof have higher affinity, indicating that all antibodies obtained by means of mutation on the basis of mutation 1 and according to the mutation manner in Table 3 have higher affinity.

(b) On the basis of VT, and other sites were mutated; the affinity of the mutants were detected with the affinity determination method of 2(a). The sequences of mutations were shown in Table 5 below, and corresponding affinity data were shown in Table 6.

TABLE 5
Mutation performed using WT as a framework

| | CDR-VH1 | CDR-VH2 | CDR-VH3 | CDR-VL1 | CDR-VL2 | CDR-VL3 |
|---|---|---|---|---|---|---|
| | X2/X3 | X2/X3/X4 | X2 | X1/X2 | X1/X2 | X1/X2 |
| WT | T/A | A/L/L | A | I/D | D/V | H/D |
| WT1 | T/G | G/L/I | V | I/N | D/V | Q/D |
| WT2 | S/A | G/V/L | A | L/N | D/V | N/D |
| WT3 | S/G | G/L/L | A | V/N | A/V | Q/D |
| WT4 | S/G | A/L/L | V | L/D | D/L | Q/E |
| WT5 | S/G | G/I/L | V | L/D | D/L | H/E |
| WT6 | T/A | A/L/I | A | L/D | A/L | H/D |
| WT7 | T/G | G/I/L | V | V/D | A/I | N/E |

TABLE 6
Affinity detection results of the WT antibody and mutants thereof

| | K_{D}(M) | kon(1/Ms) | kdis(1/s) |
|---|---|---|---|
| WT | 8.03E−09 | 1.75E+05 | 1.41E−03 |
| WT1 | 5.33E−09 | 1.35E+05 | 7.19E−04 |
| WT2 | 2.12E−09 | 3.71E+05 | 7.87E−04 |
| WT3 | 2.60E−09 | 2.78E+05 | 7.22E−04 |
| WT4 | 4.01E−09 | 2.29E+05 | 9.18E−04 |
| WT5 | 2.90E−09 | 3.28E+05 | 9.50E−04 |
| WT6 | 4.92E−09 | 1.58E+05 | 7.78E−04 |
| WT7 | 3.55E−09 | 2.37E+05 | 8.41E−04 |

The data in Table 6 showed that the WT and series mutants thereof have desirable affinity for the antigen, indicating that all antibodies obtained by means of mutation on the basis of the WT and according to the mutation manner in Table 5 have desirable affinity.

(3) Naked Antibody Stability Assessment

The above antibodies were placed at 4° C. (refrigerator), −80° C. (refrigerator) and 37° C. (incubator) for 21 days; samples at day 7, day 14 and day 21 were taken for state observation, and the samples at day 21 were detected for activity. The results showed that the antibodies did not show any obvious protein state change under three assessment conditions for 21 days, and the activity did not show a decreasing trend with the rising of an assessment temperature, indicating that the above antibodies are stable. Table 7 below showed OD results of the enzyme immunoassay activity assay for the mutation 1 antibody with 21 days of assessment.

TABLE 7

| Sample concentration (ng/ml) | 500 | 250 | 0 |
|---|---|---|---|
| 4° C., samples at day 21 | 1.189 | 0.951 | 0.030 |
| −80° C., samples at day 21 | 1.250 | 0.998 | 0.033 |
| 37° C., samples at day 21 | 1.165 | 0.905 | 0.047 |

The above are only the preferred embodiments of the disclosure and are not intended to limit the disclosure. For those skilled in the art, the disclosure may have various modifications and variations. Any modifications, equivalent replacements, improvements and the like made within the spirit and principle of the disclosure shall fall within the scope of protection of the disclosure.

### INDUSTRIAL APPLICABILITY

The present disclosure provides an antibody against a novel coronavirus, and a reagent and a kit for detecting the novel coronavirus. The antibody may specifically bind to an N protein of the novel coronavirus, has high affinity for the N protein, and has good sensitivity and specificity for detecting the novel coronavirus. According to the present disclosure, a wider range of antibody options are provided for detection of the novel coronavirus. In addition, the detection kit provided in the present disclosure also has the same technical effect as the antibody, and has a wide application prospect and high market value.

What is claimed is:

1. An antibody or a functional fragment thereof against a novel coronavirus or an N protein thereof, comprising the following complementarity determining regions:
CDR-VH1: G-X1-T-F-S-X2-F-X3-M-H, wherein X1 is V or F, X2 is S or T, and X3 is G or A;
CDR-VH2: Y-X1-N-S-X2-S-N-X3-I-Y-Y-A-D-T-X4-K, wherein X1 is L or I, X2 is G or A, X3 is I, V or L, and X4 is I, V or L;
CDR-VH3: X1-R-H-X2-M, wherein X1 is A or T, and X2 is A or V;
CDR-VL1: S-Q-S-X1-D-Y-X2-G-D-S-X3-M, wherein X1 is I, V or L, X2 is D or N, and X3 is F or Y;
CDR-VL2: X1-A-S-N-X2-E-S, wherein X1 is A or D, and X2 is I, V or L; and
CDR-VL3: Q-X1-S-N-E-X2-P-Y, wherein X1 is N, H or Q, and X2 is D or E.

2. The antibody or the functional fragment thereof according to claim 1, wherein
in CDR-VH1, X1 is F;
in CDR-VH2, X1 is I;
in CDR-VH3, X1 is A; and
in CDR-VL1, X3 is Y, wherein
preferably, in CDR-VH1, X2 is S;
preferably, in CDR-VH1, X2 is T;
preferably, in CDR-VH1, X3 is G;
preferably, in CDR-VH1, X3 is A;
preferably, in CDR-VH2, X2 is G;
preferably, in CDR-VH2, X2 is A;
preferably, in CDR-VH2, X3 is I;
preferably, in CDR-VH2, X3 is V;
preferably, in CDR-VH2, X3 is L;
preferably, in CDR-VH2, X4 is I;
preferably, in CDR-VH2, X4 is V;
preferably, in CDR-VH2, X4 is L;
preferably, in CDR-VH3, X2 is A;
preferably, in CDR-VH3, X2 is V;
preferably, in CDR-VL1, X1 is I;
preferably, in CDR-VL1, X1 is V;
preferably, in CDR-VL1, X1 is L;
preferably, in CDR-VL1, X2 is D;
preferably, in CDR-VL1, X2 is N;
preferably, in CDR-VL2, X1 is A;
preferably, in CDR-VL2, X1 is D;
preferably, in CDR-VL2, X2 is I;
preferably, in CDR-VL2, X2 is V;
preferably, in CDR-VL2, X2 is L;
preferably, in CDR-VL1, X1 is N;
preferably, in CDR-VL1, X1 is H;
preferably, in CDR-VL1, X1 is Q;
preferably, in CDR-VL2, X2 is D;
preferably, in CDR-VL2, X2 is E; and
preferably, each complementarity determining region of the antibody or the functional fragment thereof is selected from any one of the following mutation combinations 1-68:

| | CDR-VH1 | CDR-VH2 | CDR-VH3 | CDR-VL1 | CDR-VL2 | CDR-VL3 |
|---|---|---|---|---|---|---|
| | X2/X3 | X2/X3/X4 | X2 | X1/X2 | X1/X2 | X1/X2 |
| Mutation combination 1 | T/A | A/L/L | A | I/D | D/V | H/D |
| Mutation combination 2 | T/G | A/V/I | V | V/D | D/L | H/E |
| Mutation combination 3 | S/A | A/I/V | V | L/D | D/I | N/D |
| Mutation combination 4 | S/G | G/V/L | A | I/N | A/V | N/E |
| Mutation combination 5 | T/A | G/I/I | V | V/N | A/L | Q/D |
| Mutation combination 6 | S/G | A/I/V | A | L/N | A/I | Q/E |
| Mutation combination 7 | T/G | G/L/L | A | I/N | A/L | Q/D |
| Mutation combination 8 | T/A | G/V/L | V | V/D | D/V | N/D |
| Mutation combination 9 | S/A | A/L/V | V | L/N | A/I | H/D |
| Mutation combination 10 | T/A | G/I/L | A | I/D | D/L | Q/E |
| Mutation combination 11 | T/A | A/L/V | A | V/N | A/V | N/E |
| Mutation combination 12 | T/G | A/L/I | V | L/D | D/I | H/E |
| Mutation combination 13 | T/G | A/I/I | V | I/D | D/L | Q/E |
| Mutation combination 14 | T/G | A/L/V | A | L/N | A/V | H/D |
| Mutation combination 15 | T/G | G/L/L | A | V/D | D/V | N/E |
| Mutation combination 16 | S/G | G/L/L | V | V/N | A/L | Q/D |
| Mutation combination 17 | S/G | G/V/L | V | L/D | D/I | H/E |
| Mutation combination 18 | S/A | G/L/I | A | I/N | A/I | N/D |
| Mutation combination 19 | T/G | G/I/I | A | V/D | D/I | N/D |
| Mutation combination 20 | T/G | G/V/V | V | I/N | D/L | Q/E |
| Mutation combination 21 | S/A | A/L/V | V | L/D | D/V | H/D |
| Mutation combination 22 | T/G | G/V/L | A | V/N | A/I | N/E |
| Mutation combination 23 | S/A | A/V/L | A | I/D | A/L | Q/D |
| Mutation combination 24 | T/G | A/L/L | V | L/N | A/V | H/E |
| Mutation combination 25 | S/G | A/V/I | V | L/D | D/L | Q/E |
| Mutation combination 26 | T/G | G/V/V | A | I/N | A/L | N/D |
| Mutation combination 27 | T/G | A/I/L | A | V/D | D/V | H/E |
| Mutation combination 28 | S/A | G/L/I | V | L/N | A/V | Q/D |
| Mutation combination 29 | S/G | G/V/I | V | I/D | D/I | N/E |
| Mutation combination 30 | T/A | A/V/V | A | V/N | A/I | H/D |
| Mutation combination 31 | T/A | G/L/V | A | I/D | A/V | N/D |
| Mutation combination 32 | T/G | G/V/I | V | L/N | D/L | N/E |
| Mutation combination 33 | T/A | G/I/I | V | V/D | A/L | H/D |
| Mutation combination 34 | S/G | G/I/V | A | V/N | D/I | H/E |
| Mutation combination 35 | T/A | G/L/I | A | L/D | A/I | Q/D |
| Mutation combination 36 | S/G | G/L/V | V | I/N | D/V | Q/E |
| Mutation combination 37 | S/G | A/I/I | V | I/N | D/I | N/E |
| Mutation combination 38 | T/G | G/I/V | A | V/D | D/L | Q/D |
| Mutation combination 39 | T/G | G/V/L | A | L/N | D/V | H/E |
| Mutation combination 40 | T/G | G/L/L | V | I/D | A/I | N/D |
| Mutation combination 41 | T/A | G/L/L | V | V/N | A/L | Q/E |
| Mutation combination 42 | S/G | G/I/V | A | L/D | A/V | H/D |
| Mutation combination 43 | T/A | A/I/I | A | I/D | A/L | Q/E |
| Mutation combination 44 | S/G | G/V/L | V | I/N | D/V | H/D |
| Mutation combination 45 | T/A | A/V/V | V | L/D | A/I | N/E |
| Mutation combination 46 | S/A | A/I/L | A | L/N | D/L | Q/D |
| Mutation combination 47 | S/G | G/I/V | A | V/D | A/V | H/E |
| Mutation combination 48 | S/A | G/V/V | V | V/N | D/I | N/D |
| Mutation combination 49 | T/G | A/I/L | V | L/D | D/I | N/D |
| Mutation combination 50 | S/A | G/I/V | A | I/N | D/L | Q/E |
| Mutation combination 51 | S/A | G/V/V | A | V/D | D/V | H/D |
| Mutation combination 52 | S/G | A/I/I | V | L/N | A/I | N/E |
| Mutation combination 53 | T/A | A/L/V | V | I/D | A/L | Q/D |
| Mutation combination 54 | T/A | A/I/I | A | V/N | A/V | H/E |
| Mutation combination 55 | S/A | A/V/L | A | L/N | D/L | Q/E |
| Mutation combination 56 | T/G | G/V/V | V | L/D | A/V | N/D |
| Mutation combination 57 | S/A | A/V/I | V | V/N | D/V | H/E |
| Mutation combination 58 | T/G | A/I/V | A | V/D | A/L | Q/D |
| Mutation combination 59 | S/G | A/I/I | A | I/N | D/I | N/E |
| Mutation combination 60 | T/G | G/V/I | V | I/D | A/I | H/D |
| Mutation combination 61 | S/G | G/L/V | V | V/N | A/V | Q/E |
| Mutation combination 62 | T/A | A/L/V | A | L/D | D/L | Q/D |
| Mutation combination 63 | T/G | G/I/V | V | I/N | A/V | N/D |
| Mutation combination 64 | S/G | A/V/I | V | V/D | D/V | H/D |
| Mutation combination 65 | T/A | G/L/V | A | L/N | A/L | Q/E |
| Mutation combination 66 | S/G | A/I/I | A | I/D | D/I | N/E |
| Mutation combination 67 | T/G | G/V/I | V | V/N | A/I | H/E |
| Mutation combination 68 | T/G | A/I/V | A | L/D | D/L | Q/D |

3. The antibody or the functional fragment thereof according to claim 2, wherein the antibody or the functional fragment thereof binds to the N protein of the novel coronavirus with an affinity of K_{D}≤8×10⁻⁹ mol/L; and preferably, K_{D}≤7×10⁻¹⁰ mol/L.

4. The antibody or the functional fragment thereof according to claim 1, wherein
in CDR-VH1, X1 is V;
in CDR-VH2, X1 is L;
in CDR-VH3, X1 is T;
in CDR-VL1, X3 is F; and
preferably, each complementarity determining region of the antibody or the functional fragment thereof is selected from any one of the following mutation combinations 69-76:

| | CDR-VH1 | CDR-VH2 | CDR-VH3 | CDR-VL1 | CDR-VL2 | CDR-VL3 |
|---|---|---|---|---|---|---|
| | X2/X3 | X2/X3/X4 | X2 | X1/X2 | X1/X2 | X1/X2 |
| Mutation combination 69 | T/A | A/L/L | A | I/D | D/V | H/D |
| Mutation combination 70 | T/G | G/L/I | V | I/N | D/V | Q/D |
| Mutation combination 71 | S/A | G/V/L | A | L/N | D/V | N/D |
| Mutation combination 72 | S/G | G/L/L | A | V/N | A/V | Q/D |
| Mutation combination 73 | S/G | A/L/L | V | L/D | D/L | Q/E |
| Mutation combination 74 | S/G | G/I/L | V | L/D | D/L | H/E |
| Mutation combination 75 | T/A | A/L/I | A | L/D | A/L | H/D |
| Mutation combination 76 | T/G | G/I/L | V | V/D | A/I | N/E |

5. The antibody or the functional fragment thereof according to claim 1, wherein the antibody comprises light chain framework regions FR1-L, FR2-L, FR3-L and FR4-L, of which sequences are successively shown as SEQ ID NOs:1-4 or have at least 80% identity to thereof, and/or heavy chain framework regions FR1-H, FR2-H, FR3-H and FR4-H, of which sequences are successively shown as SEQ ID NOs:5-8 or have at least 80% identity to thereof;
preferably, an amino acid sequence of FR1-H is shown as SEQ ID NO:15;
preferably, the antibody further comprises a constant region;
preferably, the constant region is selected from a constant region of any one of IgG1, IgG2, IgG3, IgG4, IgA, IgM, IgE and IgD;
preferably, the species source of the constant region is cattle, horse, dairy cow, pig, sheep, goat, rat, mouse, dog, cat, rabbit, camel, donkey, deer, mink, chicken, duck, goose, turkey, gamecock or human;
preferably, the constant region is derived from mouse;
preferably, a sequence of a light chain constant region of the constant region is shown as SEQ ID NO:9 or has at least 80% identity to thereof, and a sequence of a heavy chain constant region of the constant region is shown as SEQ ID NO:10 or has at least 80% identity to thereof;
preferably, a sequence of the heavy chain constant region is shown as SEQ ID NO:16; and
preferably, the functional fragment is selected from any one of VHH, F(ab′)2, Fab′, Fab, Fv and scFv of the antibody.

6. (canceled)

7. A reagent or a kit for detecting a novel coronavirus or an N protein thereof, comprising the antibody or the functional fragment thereof according to claim 1.

8. The reagent or the kit according to claim 7, wherein the antibody or the functional fragment thereof is labeled with a detectable marker;
preferably, the detectable marker is selected from a fluorescent dye, an enzyme that catalyzes color development of substrates, a radio isotope, a chemiluminescence reagent and a nanoparticle marker;
preferably, the fluorescent dye is selected from fluorescein dyes and derivatives thereof, rhodamine dyes and derivatives thereof, Cy series dyes and derivatives thereof, Alexa series dyes and derivatives thereof, and protein dyes and derivatives thereof;
preferably, the enzyme that catalyzes color development of substrates is selected from horseradish peroxidase, alkaline phosphatase, β-galactosidase, glucose oxidase, carbonic anhydrase, acetylcholinesterase and glucose glucose 6-phosphate dehydrogenase;
preferably, the radio isotope is selected from ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ⁹⁴mTc, ⁹⁹mTc, ²⁰³Pb, ⁶⁷Ga, ⁶⁸Ga, ⁴³Sc, ⁴⁷Sc, ¹¹⁰mIn, ⁹⁷Ru, ⁶²Cu, ⁶⁴Cu, ⁶⁷Cu, ⁶⁸Cu, ⁸⁶Y, ⁸⁸Y, ¹²¹Sn, ¹⁶¹Tb, ¹⁶⁶Ho, ¹⁰⁵Rh, ¹⁷⁷Lu, ¹⁷²Lu and ¹⁸F;
preferably, the chemiluminescence reagent is selected from luminol and derivatives thereof, lucigenin, a crustacean fluorescein and derivatives thereof, a bipyridine ruthenium and derivatives thereof, an acridinium ester and derivatives thereof, a dioxycyclohexane and derivatives thereof, lophine and derivatives thereof, and a peroxyoxalate and derivatives thereof;
preferably, the nanoparticle marker is selected from nanoparticles, colloids, organic nanoparticles, magnetic nanoparticles, quantum dot nanoparticles and rare earth complex nanoparticles;
preferably, the colloids are selected from colloidal metals, disperse dyes, dye labeled microspheres, and latexes; and
preferably, the colloidal metals are selected from colloidal gold, colloidal silver and colloidal selenium.

9. A nucleic acid molecule, encoding the antibody or the functional fragment thereof according to claim 1.

10. A vector, comprising a nucleic acid fragment encoding the antibody or the functional fragment thereof according to claim 1.

11. A recombinant cell, comprising the vector according to claim 10.

12. (canceled)

13. Use of the antibody or the functional fragment thereof according to claim 1 or a reagent or a kit comprising the antibody or the functional fragment thereof according to claim 1 for detecting a novel coronavirus.

14. A method for detecting a novel coronavirus, comprising:
A) under conditions sufficient for a binding reaction to occur, contacting the antibody or the functional fragment thereof according to claim 1 with a sample, so as to perform the binding reaction; and
B) detecting an immune complex produced by the binding reaction.

15. A method for diagnosing a subject in a novel coronavirus infection or a disease associated with the novel coronavirus infection, comprising:
A) under conditions sufficient for a binding reaction to occur, contacting the antibody or the functional fragment thereof according to claim 1 with a sample from the subject, so as to perform the binding reaction; and
B) detecting an immune complex produced by the binding reaction.

16. The method according to claim 15, wherein the disease associated with the novel coronavirus infection comprises respiratory symptoms, fever, cough, shortness of breath, difficult breathing, pneumonia, severe acute respiratory syndrome, and renal failure.

17. The method according to claim 15, wherein the subject in the novel coronavirus infection comprises an asymptomatic patient, an infected patient without obvious symptoms, and a symptomatic infected patient.

18. A method for preparing the antibody or the functional fragment thereof according to claim 1, comprising: culturing a recombinant cell comprising a vector comprising a nucleic acid fragment encoding the antibody or the functional fragment thereof according to claim 1, and obtaining the antibody or the functional fragment from a culture product by means of separation and purification.
